(19) United States
(12) Patent Application Publication
(10) Pub. No.: US 2008/0114028 A1
(43) Pub. Date: May 15, 2008

(54) Process for preparing polymorphic forms of solifenacin succinate

(76) Inventors: Tamas Koltai, Netanya (IL); Tamar Nidam, Yehud (IL); Eyal Gilboa, Bat-Yam (IL); Nurit Perlman, Kfar Saba (IL); Michael Pinhasov, Dover, NJ (US); Mili Abramov, Givataim (IL)

Correspondence Address: KENYON & KENYON LLP, ONE BROADWAY, NEW YORK, NY, 10004, US

(21) Appl. No.: 11/881,161

(22) Filed: Jul. 24, 2007

Related U.S. Application Data
(60) Provisional application No. 60/833,542, filed on Jul. 24, 2006.
(60) Provisional application No. 60/846,192, filed on Sep. 20, 2006.
(60) Provisional application No. 60/861,420, filed on Nov. 29, 2006.
(60) Provisional application No. 60/924,787, filed on May 31, 2007.
(60) Provisional application No. 60/924,902, filed on Jun. 5, 2007.

(51) Int. Cl. A61K 31/4745; C07D 453/02

(52) U.S. Cl. 514/305; 546/133

(57) ABSTRACT

Polymorphic forms of solifenacin have been prepared and characterized. These polymorphic forms are particularly useful in pharmaceutical compositions.

### CROSS-REFERENCE TO RELATED APPLICATIONS

This application claims the benefit of Provisional Application Ser. No. 60/833,542, filed Jul. 24, 2006, Provisional Application Ser. No. 60/846,192, filed Sep. 20, 2006, Provisional Application Ser. No. 60/861,420, filed Nov. 29, 2006, Provisional Application Ser. No. 60/924,787, filed May 31, 2007, and to Provisional Application Ser. No. 60/924,902, filed Jun. 5, 2007. The contents of these applications are incorporated herein in their entirety by reference.

### FIELD OF THE INVENTION

The invention is directed to polymorphic forms of solifenacin succinate and processes for their preparation.

### BACKGROUND OF THE INVENTION

(3R)-1-Azabicyclo[2.2.2]oct-3-yl-(1S)-1-phenyl-3,4-dihydroisoquinoline-2-(1H)-carboxylate (1(S)-Phenyl-1,2,3,4-tetrahydroisoquinoline-2-carboxylic acid 3(R)-quinuclidinyl ester) is known as solifenacin, also known as YM-905 (in its free base form) and YM-67905 (in its succinate form). Solifenacin has the molecular formula: C₂₃H₂₆O₂, a molecular weight of 362.4647 and the following chemical structure:

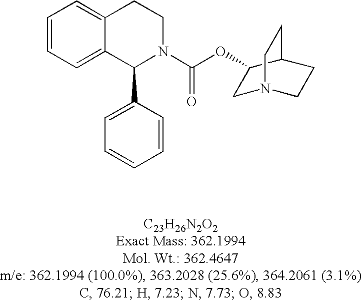

Solifenacin succinate is a urinary antispasmodic, acting as a selective antagonist to the M(3)-receptor. It is used for the treatment of symptoms of overactive bladder, such as urinary urgency and increased urinary frequency, as may occur in patients with overactive bladder syndrome (“OAB”). See Chilman-Blair, Kim et al. Drugs of Today 40(4):343-353 (2004). Solifenacin succinate is reported to be a white to pale yellowish-white crystalline powder, which is freely soluble at room temperature in water, glacial acetic acid, dimethylsulfoxide, and methanol.

Solifenacin succinate is currently marketed under the trade name VESICARE®. VESICARE® has been approved by the FDA for once daily treatment of OAB and is prescribed as 5 mg and 10 mg tablets.

Solifenacin succinate was reportedly developed by Yamanouchi Pharmaceutical Co. Ltd. and is disclosed in U.S. Pat. No. 6,017,927 (“'927 patent”) and U.S. Pat. No. 6,174,896 (“'896 patent”).

Publication IPCOM000147748D disclosed a crystalline form characterized by powder X-ray diffraction (“PXRD”) peaks at about 3.9, 11.2, 14.3, and 18.8°±0.2° 2θ, and may be further characterized by PXRD peaks at about 7.6, 19.3, 21.1, 23.2, and 25.2°±0.2° 2θ. The crystalline form may also be characterized by a PXRD pattern substantially as depicted in FIG. 1.

The present invention encompasses the solid state physical properties of solifenacin succinate. These properties can be influenced by controlling the conditions under which solifenacin succinate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally administered active ingredient can reach the patient's bloodstream. The rate of dissolution is also a consideration in formulating syrups, elixirs, and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (“TGA”), and differential scanning calorimetry (“DSC”), and can be used to distinguish some polymorphic forms from others. A particular polymorphic form may also give rise to distinct spectroscopic properties that may be detectable by powder X-ray crystallography, solid state ¹³C NMR spectrometry, and infrared spectrometry.

One of the most important physical properties of a pharmaceutical compound that can form polymorphs or solvates is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

### SUMMARY OF THE INVENTION

In one embodiment, the invention encompasses a process for preparing the crystalline form of solifenacin succinate characterized by PXRD peaks at about 3.9, 11.2, 14.3, and 18.8°±0.2° 2θ (denominated “Form I”) comprising combining solifenacin, succinic acid, and a solvent selected from the group consisting of C₃-C₅ carbonate, acetonitrile, dimethoxypropane, C₆-C₉ aromatic hydrocarbon, diethyl ether, diisopropyl ether, C₅-C₉ ester, C₁-C₄ alcohol, C₃-C₉ ketone, cyclohexane, heptane, and mixtures thereof to obtain a precipitate of the crystalline form.

In one embodiment, the invention also encompasses a process for preparing the crystalline form of solifenacin succinate characterized by PXRD peaks at about 3.9, 11.2, 14.3, and 18.8°±0.2° 2θ, comprising slurrying amorphous solifenacin succinate in a solvent selected from the group consisting of heptane, petroleum ether, cyclohexane, methyl t-butyl ether, ethyl acetate, methyl isobutylketone, CCl₄, toluene, diethyl carbonate, ethyl lactate, isobutyl acetate, methylethylketone, diethyl ether, isopropanol, dimethyl carbonate, and mixtures thereof.

In one embodiment, the invention also encompasses a process for preparing the crystalline form of solifenacin succinate characterized by PXRD peaks at about 3.9, 11.2, 14.3, and 18.8°±0.2° 2θ, comprising combining solifenacin succinate with a solvent selected from the group consisting of ethanol, methanol, 1-propanol, tetrahydrofuran, dioxane, ethyl lactate, dichloromethane, 1,2-dichloroethane, acetonitrile, dimethylacetamide A, dimethylformamide, t-butanol, 2-butanol, isopropanol, methylethylketone, toluene, CCl₄, methyl t-butyl ether, di-isopropyl ether, methyl acetate, ethyl acetate, acetone, isopropylmethyl ketone, and mixtures thereof to form a solution.

In one embodiment, the invention also encompasses a process for preparing a crystalline form of solifenacin succinate characterized by PXRD peaks at about 3.9, 11.2, 14.3, and 18.8°±0.2° 2θ, comprising exposing solifenacin succinate to solvent vapors, wherein the solvent is selected from the group consisting of cyclohexane, isopropanol, ethanol, n-butanol, diethyl ether, methyl t-butyl ether, ethyl acetate, butyl acetate, acetone, methyl isobutylketone, toluene, isopropylether, methyl acetate, 1-propanol, 2-butanol, acetonitrile, tetrahydrofuran, and mixtures thereof.

In one embodiment, the invention encompasses a process for preparing a crystalline form of solifenacin succinate characterized by PXRD peaks at about 3.9, 11.2, 14.3, and 18.8°±0.2° 2θ, comprising combining succinic acid and solifenacin.

In one embodiment, the invention encompasses a crystalline form of solifenacin succinate (denominated “Form II”) characterized by PXRD peaks at about 4.3, 14.7, and 16.2°±0.2° 2θ. The crystalline form may be further characterized by PXRD peaks at about 11.7, 18.3, 19.9, 22.3, 23.7 and 25.6°±0.2°2θ. The crystalline form may also be characterized by a PXRD pattern substantially as depicted in FIG. 4.

In one embodiment, the invention encompasses a crystalline form of solifenacin succinate characterized by PXRD peaks at about 4.3, 14.7, and 16.2°±0.2° 2θ contains a total weight of not more than about 10 wt %, more preferably not more than about 5 wt % and most preferably not more than about 2 wt % of crystalline solifenacin succinate. (For example, if a sample contains Form A and Form B as impurities, the total weight of Form A plus Form B is not more than about 10%).

In another embodiment, a crystalline form of solifenacin succinate characterized by PXRD peaks at about 4.3, 14.7, and 16.2°±0.2° 2θ contains not more than about 10 wt %, preferably not more than about 5 wt % and more preferably not more than about 2 wt % of the crystalline form of solifenacin succinate characterized by PXRD peaks at about 3.9, 11.2, 14.3, and 18.8°±0.2° 2θ. The amount of the crystalline form of solifenacin succinate characterized by PXRD peaks at about 3.9, 11.2, 14.3, and 18.8°±0.2° 2θ present may be calculated based on area percentages of the PXRD peaks at 7.6, 11.2, 21.9 and 25.2±0.2° 2θ.

In another embodiment, a crystalline form of solifenacin succinate characterized by PXRD peaks at about 4.3, 14.7, and 16.2°±0.2° 2θ contains not more than about 10 wt %, more preferably not more than about 5 wt %, most preferably not more than about 2 wt %, of any other crystalline form. (For example, if a sample contains Form A and Form B as impurities, Form A and Form B are each present in not more than about 10%). The weight percentages may be calculated based on area percentages of the PXRD peaks.

In one embodiment, the invention also encompasses a process for preparing the crystalline form of solifenacin succinate characterized by PXRD peaks at about 4.3, 14.7, and 16.2°±0.2° 2θ, comprising combining solifenacin with a solvent selected from methyl acetate and methyl t-butyl ether to obtain a solution, adding succinic acid to obtain a slurry containing solifenacin succinate, and optionally recovering the crystalline solifenacin succinate.

In one embodiment, the invention also encompasses a process for preparing a crystalline form of solifenacin succinate characterized by PXRD peaks at about 4.3, 14.7, and 16.2°±0.2° 2θ, comprising slurrying amorphous solifenacin succinate in a solvent selected from n-butanol and isobutyl acetate.

In one embodiment, the invention also provides an amorphous form of solifenacin succinate.

In one embodiment, the invention also provides an amorphous form of solifenacin succinate having not more than about 10 wt %, preferably not more than about 5 wt %, more preferably not more than about 1 wt % of crystalline solifenacin succinate. In another embodiment, an amorphous form of solifenacin succinate contains not more than about 10 wt %, preferably not more than about 5 wt %, more preferably not more than about 1 wt % of the crystalline form of solifenacin succinate characterized by PXRD peaks at about 3.9, 11.2, 14.3, and 18.8°±0.2° 2θ. The crystalline weight percentages may be calculated based on area percentages of the PXRD peaks. The amorphous form of solifenacin succinate may be characterized by a PXRD pattern substantially as depicted in FIG. 2.

In one embodiment, the invention also encompasses processes for preparing amorphous solifenacin succinate comprising dissolving solifenacin succinate in methanol or water and spray drying the solution to obtain amorphous solifenacin succinate.

In one embodiment, the invention encompasses a process for preparing amorphous solifenacin succinate comprising dissolving solifenacin succinate in water and lyophilizing it to obtain amorphous solifenacin succinate.

In one embodiment, the invention also encompasses a pharmaceutical composition comprising at least one of an amorphous form of solifenacin succinate and a crystalline form of solifenacin succinate characterized by PXRD peaks at about 4.3, 14.7, and 16.2°±0.2° 2θ, and at least one pharmaceutically acceptable excipient.

In one embodiment, the invention also encompasses a process for preparing a pharmaceutical composition comprising combining at least one of an amorphous form of solifenacin succinate and a crystalline form of solifenacin succinate characterized by PXRD peaks at about 4.3, 14.7, and 16.2°±0.2° 2θ with at least one pharmaceutically acceptable excipient.

In one embodiment, the invention also encompasses the use of at least one of an amorphous form of solifenacin succinate and a crystalline form of solifenacin succinate characterized by PXRD peaks at about 4.3, 14.7, and 16.2°±0.2° 2θ in the manufacture of a medicament for the treatment of overactive bladder syndrome.

In one embodiment, the invention also encompasses a method of treatment of overactive bladder syndrome comprising administering a pharmaceutical composition comprising at least one of an amorphous form of solifenacin succinate and a crystalline form of solifenacin succinate characterized by PXRD peaks at about 4.3, 14.7, and 16.2°±0.2° 2θ, and at least one pharmaceutically acceptable excipient to a patient in need thereof.

### BRIEF DESCRIPTION OF THE FIGURES

FIG. 1 illustrates a PXRD pattern of solifenacin succinate crystalline form characterized by PXRD peaks at about 3.9, 11.2, 14.3, and 18.8°±0.2° 2θ.

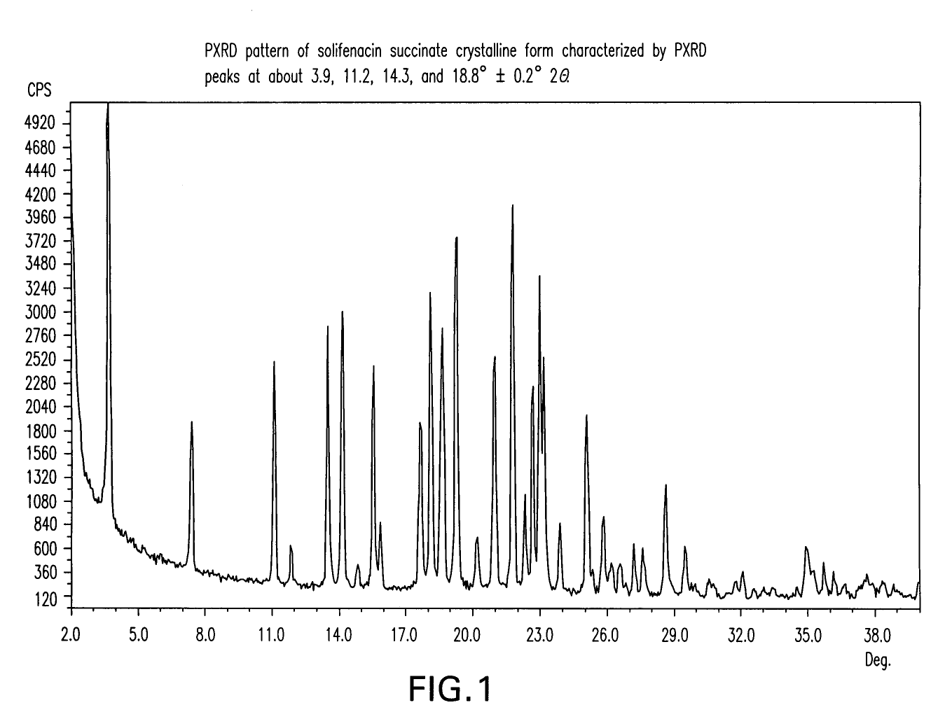

FIG. 2 illustrates a PXRD pattern for amorphous solifenacin succinate made according to Example 25.

FIG. 3 illustrates a PXRD pattern for amorphous solifenacin succinate made according to Example 26.

FIG. 4 illustrates a PXRD pattern of solifenacin succinate crystalline form characterized by PXRD peaks at about 4.3, 14.7, and 16.2°±0.2° 2θ.

### DETAILED DESCRIPTION OF THE INVENTION

The invention addresses the need in the art for new solid forms of solifenacin succinate by providing crystalline and amorphous forms of solifenacin succinate.

As used herein, the term “room temperature” (RT) means the ambient temperature of a typical laboratory, which is usually about 15° C. to about 30° C., often about 18° C. to about 25° C.

As used herein, the term “reflux temperature” refers to the boiling point of the solvent.

As used herein, the term “lyophilization” refers to a freeze drying process.

As used herein, the term “vacuum” refers to a pressure of about to 2 mmHg to about 50 mm Hg.

As used herein, the term “PXRD” refers to powder X-ray diffraction,

As used herein, the term “ACN” refers to acetonitrile, the term “ACN” refers to acetonitrile, the term “EtOH” refers to ethanol, the term “MeOH” refers to methanol, the term “BuOH” refers to butanol, the term “THF” refers to tetrahydrofuran, the term “DCM” refers to dichloromethane, the term “DMA” refers to dimethylacetamide, the term “DMF” refers to dimethylformamide, the term “DEE” refers to diethyl ether, the term “MTBE” refers to methyl t-butyl ether, the term “EtOAc” refers to ethyl acetate, the term “MEK” refers to methylethylketone, the term “MIBK” refers to methyl isobutylketone, the term “DIPE” refers to di-isopropyl ether, the term “BuOAc” refers to butyl acetate, the term “MeOAc” refers to methyl acetate, the term “DMC” refers to dimethyl carbonate, the term “IBA” refers to isobutyl acetate, the term “DEC” refers to diethyl carbonate, the term “IPA” refers to isopropanol, the term “DMSO” refers to dimethylsulfoxide, the term “IPE” refers to isopropylether.

As used herein, the term “CDI” refers to carbonyldiimidazole, the term “IQL-Im” refers to IQL-Imidazole, the term “SLF” refers to solifenacin.

The invention encompasses a process for preparing the crystalline form of solifenacin succinate characterized by PXRD peaks at about 3.9, 11.2, 14.3, and 18.8°±0.2° 2θ comprising combining solifenacin, succinic acid, and a solvent selected from the group consisting of C₃-C₅ carbonate, ACN, dimethoxypropane, C₆-C₉ aromatic hydrocarbon, diethyl ether, diisopropyl ether, C₅-C₉ ester, C₁-C₄ alcohol, C₃-C₉ ketone, cyclohexane, heptane, and mixtures thereof to obtain a precipitate of the crystalline form.

The solifenacin starting material may be prepared according to the procedures set forth in PCT International Publication No. WO 2005/105795.

Preferably, the C₆-C₉ aromatic hydrocarbon is toluene. Preferably, the C₃-C₅ carbonate is DMC. Preferably, the C₁-C₄ alcohol is selected from the group consisting of ethanol, EPA, and n-BuOH. Preferably, the C₃-C₉ ketone is selected from the group consisting of MEK, acetone, and MIBK. Preferably, the C₅-C₉ ester is IBA. Preferably, the solvent is selected from the group consisting of ethanol, IBA, MEK, IPE, a mixture of ethanol and MEK, a mixture of ethanol and MIBK, a mixture of ethanol and acetone, MIBK, acetone, cyclohexane, and heptane.

Optionally, after succinic acid is added, a slurry containing solifenacin succinate is obtained.

In one embodiment of the invention, the solifenacin is combined with the solvent to form a solution, and succinic acid is combined with the solution to obtain a precipitate of the crystalline form.

Preferably, the process comprises combining the solifenacin and the solvent, heating, adding succinic acid, and cooling. The heating may be to a temperature of about 30° C. to about 70° C., preferably to about 40° C. to about 60° C., more preferably to about 50° C. After heating, a clear solution is obtained. Preferably, prior to the cooling step, the reaction mixture is stirred. Preferably, the stirring is for about 5 minutes to about 4 hours. The cooling is preferably to a temperature of about room temperature to about −10° C., more preferably, to about 0° C. Preferably, the cooled slurry is stirred. Preferably, the stirring is for about 2 hours to about 16 hours.

Optionally, when the solvent is selected from the group consisting of ethanol, IPA, IBA, and MEK, the solifenacin and the solvent are combined at a temperature of about 15° C. to about 30° C., more preferably at a temperature of about 20° C. to about 25° C. Preferably, after the succinic acid addition, the reaction mixture is stirred to obtain a slurry. Preferably, the stirring is for about 2 to about 24 hours, more preferably for about 4 to about 16 hours. Preferably, the stirring is at about 15° C. to about 30° C., more preferably at a temperature of about 20° C. to about 25° C.

In one embodiment of the invention, the solifenacin is combined with the succinic acid, and the solvent is added to obtain a precipitate of the crystalline form of solifenacin succinate characterized by PXRD peaks at about 3.9, 11.2, 14.3, and 18.8°±0.2° 2θ. Preferably, the solvent is selected from the group consisting of cyclohexane, heptane, and n-BuOH. Preferably, the reaction mixture is stirred prior to the solvent addition. Preferably, the stirring is for about 1 hour. Preferably, the reaction mixture is stirred after the solvent addition. Preferably, the stirring is for about 10 hours to about 24 hours.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after filtration, the precipitated crystalline solifenacin succinate is then washed with a solvent. Optionally, the washing step includes washing with the same solvent used at the beginning of the process.

The recovered crystalline solifenacin succinate may optionally be dried. Preferably, the drying is performed at a temperature of about 40° C. to about 60° C., more preferably at a temperature of about 50° C. Preferably, the drying is performed under vacuum.

The invention also encompasses a process for preparing the crystalline form of solifenacin succinate characterized by PXRD peaks at about 3.9, 11.2, 14.3, and 18.8°±0.2° 2θ, comprising slurrying amorphous solifenacin succinate in a solvent selected from the group consisting of heptane, petroleum ether, cyclohexane, MTBE, EtOAc, MIBK, CCl₄, toluene, DEC, ethyl lactate, IBA, MEK, DEE, IPA, DMC, and mixtures thereof. When the solvent is IBA, the slurry is heated to a temperature of more than about room temperature.

Optionally, after the heating step, the ratio of solvent to solifenacin succinate is from about 4:1 to about 10:1 ml/mg, preferable about 5:1 ml/mg.

Preferably, the mixture of the solifenacin succinate and the solvent is stirred at about room temperature to about 110° C., preferably at about 100° C. Preferably, the stirring is for about 0.5 hours to about 5 hours.

Optionally, after the heating step, the mixture of the solifenacin succinate and the solvent is stirred at about room temperature, preferable at about 20° C. to about 25° C. Preferably, the stirring is for about 0.5 to about 3 hours, more preferably about 1 hour.

Preferably, the solvent is removed. Optionally, the solvent is removed by decantation.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after filtration, the precipitated crystalline solifenacin succinate is then washed with a solvent. Optionally, the washing step includes washing with the same solvent used at the beginning of the process.

The recovered crystalline solifenacin succinate may optionally be dried. Preferably, the drying is performed at a temperature of about 40° C. to about 60° C., more preferably at a temperature of about 50° C. Preferably, the drying is performed under vacuum.

The invention also encompasses a process for preparing the crystalline form of solifenacin succinate characterized by PXRD peaks at about 3.9, 11.2, 14.3, and 18.8°±0.2° 2θ, comprising combining solifenacin succinate with a solvent selected from the group consisting of EtOH, MeOH, 1-propanol, THF, dioxane, ethyl lactate, DCM, 1,2-dichloroethane, ACN, DMA, DMF, t-BuOH, 2-BuOH, IPA, MEK, toluene, CCl₄, MTBE, DIPE, MeOAc, EtOAc, acetone, isopropylmethyl ketone, and mixtures thereof.

In one embodiment of the invention, the process comprises combining solifenacin succinate with a solvent selected from the group consisting of EtOH, MeOH, 1-propanol, THF, dioxane, ethyl lactate, DCM, 1,2-dichloroethane, ACN, DMA, DMF, and mixtures thereof to form a solution, and removing the solvent to obtain the crystalline solifenacin succinate.

Optionally, the ratio of solvent to solifenacin succinate is from about 5:1 to 40:1 ml/g, about 12:1 to 40:1, or about 15:1 to 40:1. A suitable ratio depends on the solubility of solifenacin succinate in the solvent and can be easily determined by one of ordinary skill in the art.

Preferably, the solvent is removed by evaporation. Preferably, the evaporation is performed by using an evaporator. Preferably, the evaporation is performed at a temperature of about 20° C. to about 80° C., about 40° C. to about 60° C., or about 50° C. Preferably, the evaporation is performed under vacuum, more preferably, under a pressure of about 2 to about 30 mmHg.

Optionally, when ethyl lactate is the solvent, a mixture of the crystalline form of solifenacin succinate characterized by PXRD peaks at about 3.9, 11.2, 14.3, and 18.8°±0.2° 2θ and the amorphous form is obtained.

In one embodiment of the invention, the process comprises combining solifenacin succinate with a solvent selected from the group consisting of MeOAc, acetone, isopropylmethyl ketone, and mixtures thereof to form a solution.

Optionally, the mixture of the solifenacin succinate and the solvent is stirred. Preferably, the stirring is at about 60° C. to about 110° C., more preferably at about 100° C. Preferably, the stirring is for about 0.25 hours to about 2 hours, preferably about 0.5 hour.

Optionally, the mixture is maintained for sufficient time to obtain a precipitate of the crystalline form. Preferably, the mixture is maintained for about 1 day. Preferably, the mixture is maintained at about 0° C. to about 25° C., preferably about 4° C.

Preferably, the solvent is removed. Optionally, the solvent is removed by decantation.

In one embodiment of the invention, the process comprises crystallizing solifenacin succinate from a solvent selected from the group consisting of t-BuOH, IPA, EtOAc, MEK, ACN, 2-BuOH, toluene, dioxane, 1-propanol, CCl₄, EtOH, THF, MTBE, acetone, DIPE, MeOAc, MeOH, DMSO, DCM, isopropylmethyl ketone, and mixtures thereof.

Optionally, when the solvent is selected from the group consisting of t-BuOH, IPA, EtOAc, MEK, ACN, 2-BuOH, toluene, dioxane, 1-propanol, CCl₄, EtOH, THF, MTBE, acetone, DIPE, MeOAc, isopropylmethylketone, and mixtures thereof, the processes comprise dissolving solifenacin succinate in the solvent, cooling the solution, and recovering the crystalline solifenacin succinate. Preferably, the dissolving step is done while heating. Preferably, the solifenacin succinate is dissolved at a temperature of about 50° C. to about 100° C. Preferably, the cooling is to a temperature of about 30° C. to about 5° C., at about room temperature, or at about 25° C. to about 20° C.

Optionally, when the solvent is selected from the group consisting of MeOH, DMSO, and DCM, the process comprises dissolving solifenacin succinate in the solvent, combining the solution with an anti-solvent selected from the group consisting of acetone, MTBE, MeOAc, IBA, 2-BuOH, MEK, IPE, CCl₄, toluene, EtOAc, and hexane, and recovering the crystalline solifenacin succinate. Preferably, the ratio between the solvent and solifenacin succinate is from about 1.5 to about 5 ml of solvent per gram of solifenacin succinate. Preferably, the ratio between the anti-solvent and the solvent is from about 12.5:1 to about 25:1 by volume. Optionally, the mixture of the solution and the anti-solvent is stirred, preferably for about 3 to about 18 hours.

Optionally, when the solvent/anti-solvent are MeOH/acetone, the solution is heated prior to being combined with the anti-solvent. Preferably, the heating is to a temperature of about 50° C. to about reflux temperature, more preferably, to about reflux temperature. Preferably, the process further comprises a cooling step prior to the recovering step. Preferably, the cooling is to a temperature of about 5° C. to about −110° C., more preferably, to a temperature of about −5° C. Preferably, the cooling is for about 2 hours to about 20 hours.

Optionally, when the solvent/anti-solvent are either one of MeOH/MeOAc, DMSO/IBA, and DCM/acetone, the anti-solvent is cooled prior to its combination with the solution. Preferably, the cooled anti-solvent is at a temperature of about 5° C. to about −10° C., more preferably, at a temperature of about −5° C.

Optionally, when the solvent/anti-solvent are either DCM/EtOAc or DCM/hexane, the added solution is at a temperature higher than room temperature.

Optionally, when the solvent is MeOH, the anti-solvent is selected from the group consisting of acetone, MTBE, and MeOAc.

Optionally, when the solvent is DMSO, the anti-solvent is selected from the group consisting of MTBE and IBA.

Optionally, when the solvent is DCM, the anti-solvent is selected from the group consisting of 2-BuOH, MEK, acetone, IPE, CCl₄, toluene, EtOAc, and hexane.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after recovery, the precipitated crystalline solifenacin succinate is then washed with a solvent. Optionally, the washing step includes washing with the same solvent used at the beginning of the process. Preferably, the solvent is ethanol.

The recovered crystalline solifenacin succinate may optionally be dried. Preferably, the drying is performed at a temperature of about 40° C. to about 60° C., more preferably at a temperature of about 50° C. Preferably, the drying is performed under vacuum.

Optionally, when the solvent is selected from the group consisting of toluene, dioxane, 1-propanol, CCl₄, THF, MTBE, DIPE, DMSO, and DCM, the crystalline form of solifenacin succinate characterized by PXRD peaks at about 3.9, 11.2, 14.3, and 18.8°±0.2° 2θ may be obtained in a wet form.

The invention also encompasses a process for preparing a crystalline form of solifenacin succinate characterized by PXRD peaks at about 3.9, 11.2, 14.3, and 18.8°±0.2° 2θ, comprising exposing amorphous solifenacin succinate to solvent vapors, wherein the solvent is selected from the group consisting of cyclohexane, IPA, EtOH, n-BuOH, DEE, MTBE, EtOAc, BuOAc, acetone, MIBK, toluene, IPE, MeOAc, 1-propanol, 2-BuOH, ACN, THF, and mixtures thereof. Preferably, the crystalline form is then isolated.

Optionally, solifenacin succinate is dissolved in MeOH prior to the solvent exposure. Preferably, when the solifenacin succinate is dissolved in MeOH, the ratio of MeOH to solifenacin succinate is about 4:1 ml/g. Preferably, when the solifenacin succinate is dissolved in MeOH, the solvent is selected from the group consisting of toluene, IPE, MeOAc, 1-propanol, 2-BuOH, ACN, and THF.

Optionally, the solifenacin succinate is exposed to solvent vapors in a closed container. Preferably, the solifenacin succinate with the solvent vapors in the container is maintained for about 10 to about 40 days, more preferably for about one month. Optionally, after maintaining, a mixture of solvent and a precipitate is obtained in the container. Preferably, the obtained solvent is removed. Preferably, the solvent is removed with pipette.

The invention also encompasses a process for preparing a crystalline form of solifenacin succinate characterized by PXRD peaks at about 3.9, 11.2, 14.3, and 18.8°±0.2° 2θ, comprising combining succinic acid and solifenacin.

Preferably, the reaction mixture is stirred. Preferably, the stirring is at about room temperature. Preferably, the stirring is for about 4 hours to about 10 hours, more preferably, for about 5 hours.

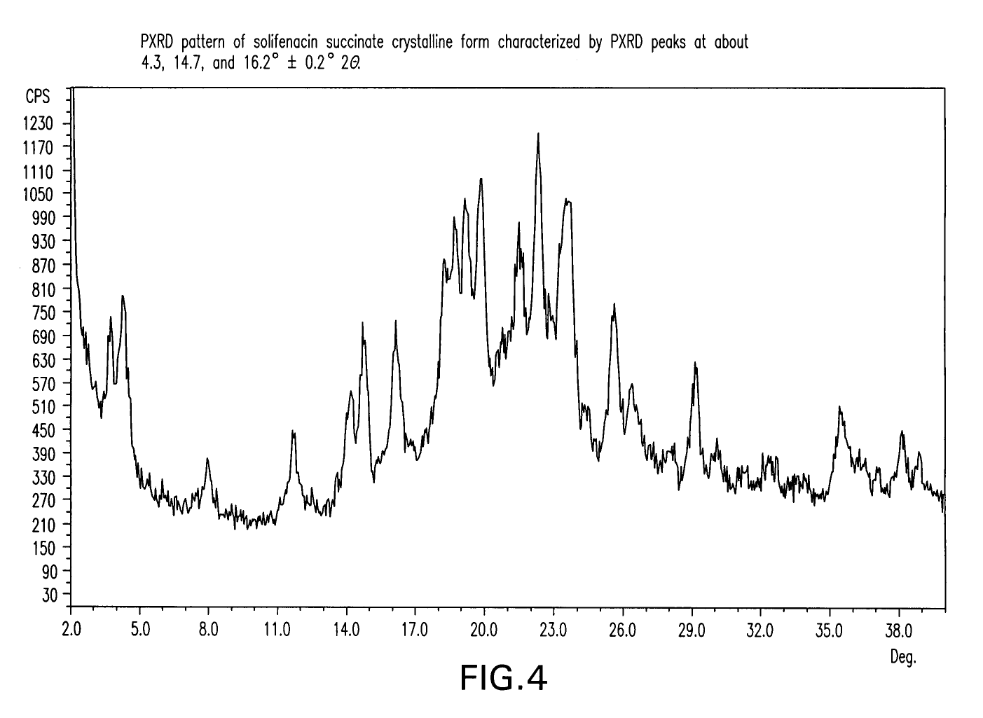

The invention encompasses a crystalline form of solifenacin succinate characterized by PXRD peaks at about 4.3, 14.7, and 16.2°±0.2° 2θ. The crystalline form may be further characterized by PXRD peaks at about 11.7, 18.3, 19.9, 22.3, 23.7 and 25.6°±0.2° 2θ. The crystalline form may also be characterized by a PXRD pattern substantially as depicted in FIG. 4. One use of this form is as an intermediate in preparing purified crystalline solifenacin succinate.

Preferably, solifenacin succinate characterized by PXRD peaks at about 4.3, 14.7, and 16.2°±0.2° 2θ contains not more than about 10 wt %, more preferably not more than about 5 wt %, most preferably not more than about 2 wt %, of any other crystalline form of solifenacin succinate. In one embodiment, solifenacin succinate characterized by PXRD peaks at about 4.3, 14.7, and 16.2°±0.2° 2θ contains not more than about 10 wt %, more preferably not more than about 5 wt %, most preferably not more than about 2 wt %, of the crystalline form of solifenacin succinate characterized by PXRD peaks at about 3.9, 11.2, 14.3, and 18.8°±0.2° 2θ. In another embodiment, a crystalline form of solifenacin succinate characterized by PXRD peaks at about 4.3, 14.7, and 16.2°±0.2° 2θ contains a total weight of all other crystalline forms of not more than about 10 wt %, more preferably not more than about 5 wt %, most preferably not more than about 2 wt %.

The invention also encompasses a process for preparing the crystalline form of solifenacin succinate characterized by PXRD peaks at about 4.3, 14.7, and 16.2°±0.2° 2θ, comprising combining solifenacin with a solvent selected from MeOAc and MTBE to obtain a solution, adding succinic acid to obtain a slurry containing solifenacin succinate, and optionally recovering the crystalline solifenacin succinate.

Preferably, the solifenacin and the solvent are combined at a temperature of about 15° C. to about 30° C., more preferably from about 20° C. to about 25° C.

Preferably, after the succinic acid addition, the reaction mixture is stirred to obtain a slurry. Preferably, the stirring is for about 2 to about 6 hours, more preferably for about 3 to about 4 hours.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after filtration, the precipitated crystalline solifenacin succinate is then washed with a solvent. Optionally, the washing step includes washing with the same solvent used at the beginning of the process.

The recovered crystalline solifenacin succinate may optionally be dried. Preferably, the drying is performed at a temperature of about 40° C. to about 60° C., more preferably at a temperature of about 50° C. Preferably, the drying is performed under vacuum.

The invention also encompasses a process for preparing a crystalline form of solifenacin succinate characterized by PXRD peaks at about 4.3, 14.7, and 16.2°±0.2° 2θ, comprising slurrying amorphous solifenacin succinate in a solvent selected from n-BuOH and IBA.

Preferably, the slurry is stirred for about 0.5 to about 3 hours. Preferably, the stirring is at about room temperature.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after filtration, the precipitated crystalline solifenacin succinate is then washed with a solvent. Optionally, the washing step includes washing with the same solvent used at the beginning of the process. Preferably, the solvent is ethanol.

The recovered crystalline solifenacin succinate may optionally be dried. Preferably, the drying is performed at a temperature of about 40° C. to about 60° C., more preferably at a temperature of about 50° C. Preferably, the drying is performed under vacuum, more preferably under a pressure of about 2 to about 30 mmHg.

The invention also encompasses a process for preparing a mixture of a crystalline form of solifenacin succinate characterized by PXRD peaks at about 4.3, 14.7, and 16.2°±0.2° 2θ and a crystalline form of solifenacin succinate characterized by PXRD peaks at about 3.9, 11.2, 14.3, and 18.8°±0.2° 2θ, comprising slurrying amorphous solifenacin succinate in a solvent selected from acetone and dioxane.

Preferably, the slurry is stirred for about 0.5 to about 3 hours. Preferably, the stirring is at about room temperature, more preferably at about 20° C. to about 25° C.

The process preferably further comprises recovering the precipitated crystalline solifenacin succinate. The precipitated crystalline solifenacin succinate may be recovered by any means known to one of skill in the art. Preferably, the precipitated crystalline solifenacin succinate is separated from the solution by filtration. Preferably, the filtration is vacuum filtration.

Preferably, after filtration, the precipitated crystalline solifenacin succinate is then washed with a solvent. Optionally, the washing step includes washing with the same solvent used at the beginning of the process. Preferably, the solvent is ethanol.

The recovered crystalline solifenacin succinate may optionally be dried. Preferably, the drying is performed at a temperature of about 40° C. to about 60° C., more preferably at a temperature of about 50° C. Preferably, the drying is performed under vacuum, more preferably under a pressure of about 2 to about 30 mmHg.

The invention also provides an amorphous form of solifenacin succinate. Generally, it has better solubility and better compressibility compared to crystalline forms.

Preferably, the amorphous form of solifenacin succinate contains not more than about 10 wt %, more preferably not more than about 5 wt %, most preferably not more than about 1 wt %, of any crystalline form of solifenacin succinate.

In another embodiment, the amorphous form of solifenacin succinate contains not more than about 10 wt %, more preferably not more than about 5 wt %, most preferably not more than about 1 wt %, of the crystalline form of solifenacin succinate characterized by PXRD peaks at about 3.9, 11.2, 14.3, and 18.8°±0.2° 2θ.

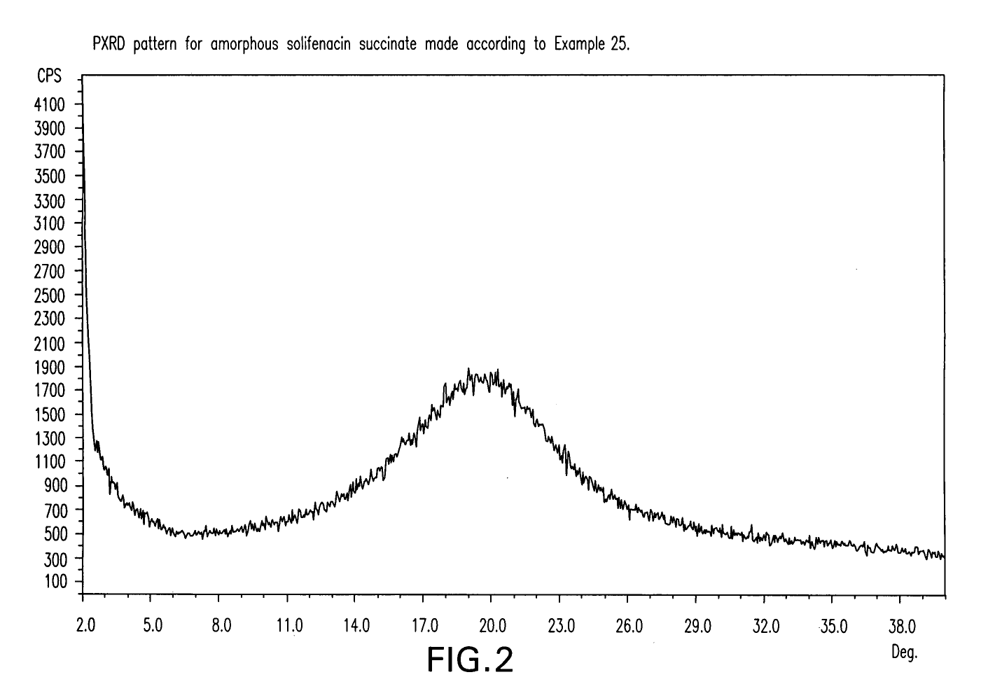

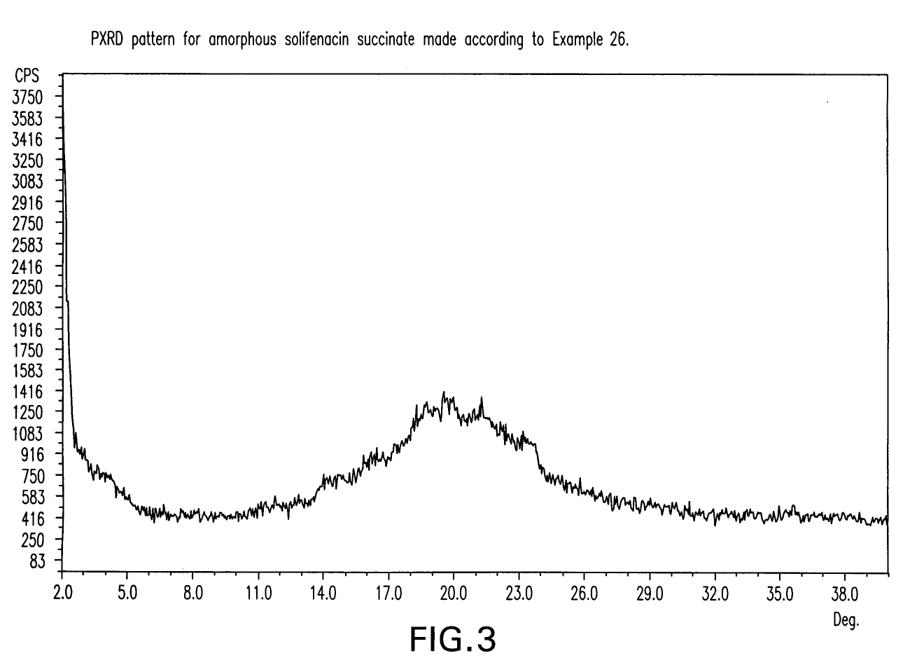

In another embodiment, the total weight of crystalline solifenacine succinate in the predominantly amorphous form of solifenacin succinate is not more than about 10 wt %, more preferably not more than about 5 wt %, most preferably not more than about 1 wt %. The crystalline weight percentages may be calculated based on area percentages of the PXRD peaks. The amorphous form of solifenacin succinate may be characterized by a PXRD pattern substantially as depicted in FIG. 2 or FIG. 3.

The invention also encompasses processes for preparing amorphous solifenacin succinate comprising dissolving solifenacin succinate in methanol or water and spray drying the solution to obtain amorphous solifenacin succinate.

Preferably, the solution is spray-dried at an inlet temperature of from about 25° C. to about 250° C., more preferably about 30° C. to about 200° C., most preferably from about 50° C. to about 150° C.

Preferably, the solution is spray-dried at an outlet temperature of from about 50° C. to about 100° C.

Preferably, when the solvent is methanol, the inlet temperature is about 150° C., and the outlet temperature is from about 92° C. to about 94° C. Preferably, when the solvent is water, the inlet temperature is about 100° C., and the outlet temperature is from about 56° C. to about 70° C.

The spray drying technique is easy to apply on an industrial scale, and avoids the thermal deterioration of the product by the very short contact time between the hot air flow and the amorphous solifenacin succinate. The process involves breaking up liquid mixtures into small droplets (atomization) and rapidly removing solvent from the mixture. The removal of the solvent is preferably by providing a drying gas. The drying gas used in the invention may be any suitable gas, although inert gases such as nitrogen, nitrogen-enriched air, and argon are preferred. Preferably, the spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream from the drying chamber. Examples of such apparatuses include Niro Models PSD-1, PSD-2, and PSD-4 (Niro A/S, Soeborg, Denmark). The solifenacin succinate product produced by spray drying may be recovered by using a cyclone or a filter. Exemplary spray drying processes and equipment are described in Perry's Chemical Engineer's Handbook, pgs. 20-54 to 20-57 (Sixth Edition, 1984), which is incorporated herein by reference.

The invention encompasses a process for preparing amorphous solifenacin succinate comprising dissolving solifenacin succinate in water and lyophilizing it to obtain amorphous solifenacin succinate.

Preferably, the lyophilization is at a temperature of about −20° C. to about −50° C.

Preferably, the lyophilization is for about 15 hours to about 30 hours. More preferably, the lyophilization is for about 20 hours.

The invention also encompasses a pharmaceutical composition comprising at least one of an amorphous form of solifenacin succinate and a crystalline form of solifenacin succinate characterized by PXRD peaks at about 4.3, 14.7 and 16.2°±0.2° 2θ, and at least one pharmaceutically acceptable excipient.

The invention also encompasses a process for preparing a pharmaceutical composition comprising combining at least one of an amorphous form of solifenacin succinate and a crystalline form of solifenacin succinate characterized by PXRD peaks at about 4.3, 14.7, and 16.2°±0.2° 2θ with at least one pharmaceutically acceptable excipient.

The invention also encompasses the use of at least one of an amorphous form of solifenacin succinate and a crystalline form of solifenacin succinate characterized by PXRD peaks at about 4.3, 14.7, and 16.2°±0.2° 2θ in the manufacture of a medicament for the treatment of overactive bladder syndrome.

The invention also encompasses a method of treatment of overactive bladder syndrome comprising administering a pharmaceutical composition comprising at least one of an amorphous form of solifenacin succinate and a crystalline form of solifenacin succinate characterized by PXRD peaks at about 4.3, 14.7, and 16.2°±0.2° 2θ, and at least one pharmaceutically acceptable excipient to a patient in need thereof.

The pharmaceutical compositions of the invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (suspensions), and the like.

Pharmaceutical compositions of the present invention can optionally be mixed with other forms of solifenacin succinate and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Carriers for use in the pharmaceutical compositions may include, but are not limited to, lactose, white sugar, sodium chloride, glucose, urea, starch, calcium carbonate, kaolin, crystalline cellulose, and silicic acid.

Binders help bind the active ingredient and other excipients together after compression. Binders for solid pharmaceutical compositions include for example acacia, alginic acid, carbomer (e.g. CARBOPOL®), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, and starch.

Disintegrants can increase dissolution. Disintegrants include, for example, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®), and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include, for example, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Glidants can be added to improve the flowability of non-compacted solid composition and improve the accuracy of dosing. Excipients that can function as glidants include, for example, colloidal silicon dioxide, magnesium trisilicate, powdered cellulose, starch, talc, and tribasic calcium phosphate.

Flavoring agents and flavor enhancers make the dosage form more palatable to the patient. Common flavoring agents and flavor enhancers for pharmaceutical products that can be included in the composition of the present invention include, for example, maltol, vanillin, ethyl vanillin, menthol, citric acid, fumaric acid, ethyl maltol, and tartaric acid.

Tablets can be further coated with commonly known coating materials. For example, the tablets can be sugar coated tablets, gelatin film coated tablets, tablets coated with enteric coatings, tablets coated with films, double layered tablets, and multi-layered tablets. Capsules can be coated with shell made, for example, from gelatin, and optionally contain a plasticizer such as glycerin and sorbitol, and an opacifying agent or colorant.

Solid and liquid compositions can also be dyed using any pharmaceutically acceptable colorant to improve their appearance and/or to facilitate patient identification of the product and unit dosage level.

In liquid pharmaceutical compositions of the present invention, the amorphous form of solifenacin succinate or the crystalline form of solifenacin succinate characterized by PXRD peaks at about 4.3, 14.7, and 16.2°±0.2° 2θ of the present invention and any other solid ingredients are dissolved or suspended in a liquid carrier such as water, vegetable oil, alcohol, polyethylene glycol, propylene glycol, and glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain viscosity enhancing agents to improve the mouth-feel of the product and/or coat the lining of the gastrointestinal tract. Such agents include, for example, acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxy toluene, butylated hydroxyanisole, and ethylenediamine tetraacetic acid can be added at safe levels to improve storage stability.

A liquid composition according to the present invention can also contain a buffer such as gluconic acid, lactic acid, citric acid or acetic acid, sodium gluconate, sodium lactate, sodium citrate, and sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended, and then further mixed in the presence of a liquid, typically water, which causes the powders to clump up into granules. The granulate is screened and/or milled, dried, and then screened and/or milled to the desired particle size. The granulate can then be tableted, or other excipients such as a glidant and/or a lubricant can be added prior to tableting.

A tableting composition can be prepared conventionally by dry blending. For instance, the blended composition of the active ingredients and excipients can be compacted into a slug or a sheet, and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only without being subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of the amorphous form of solifenacin succinate or a crystalline form of solifenacin succinate characterized by PXRD peaks at about 4.3, 14.7, and 16.2°±0.2° 2θ of the present invention contained in a pharmaceutical composition according to the present invention is not specifically restricted; however, the dose should be sufficient to treat, ameliorate, or reduce the condition.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

### Instruments

The PXRD was performed on Scintag X-ray powder diffractometer model X'TRA with a solid state detector. Copper radiation of 1.5418 Å was used. The sample holder was a round standard aluminum sample holder with rough zero background. The scanning parameters were: range: 2-40 degrees two-theta; scan mode: continuous scan; step size: 0.05 degree, and at a rate of 5 degrees/min.

### Example 1

### Procedure for the Preparation of Form I of Solifenacin Succinate

A 500 ml reactor was loaded with (S)-1-phenyl-1,2,3,4-tetrahydroquinoline (“IQL,” 46.0 g), carbonyldiimidazole (“CDI,” 39.2 g), and toluene (230 ml) to form a mixture. The mixture was stirred at room temperature for two hours, followed by the addition of CDI (2 g). After 45 minutes, the mixture was filtered, and some of the solution was evaporated to obtain a mixture of IQL-Imidazole (“IQL-Im”) in toluene.

A 1 L reactor was loaded with (R)-3-quinuclidinol (34 g), NaH (60%, 15 g), and DMF (46 ml) to form a mixture. The mixture was stirred at room temperature for 1 hour and then heated gradually to 85° C. The mixture of IQL-Im in toluene formed by the procedure in the preceding paragraph was added drop-wise over a period of 30 minutes. The reaction mixture was stirred at 85° C. for 3.75 hours, then at room temperature overnight.

The reaction mixture was then extracted with water (4×300 ml). The organic layer was separated, and the solvent was evaporated to obtain solifenacin (“SLF,” 72.6 g) as an oil.

The oil was dissolved in ethanol (350 ml) at room temperature to form a solution. Succinic acid (24 g) was added to the solution, and precipitation occurred. The precipitate was isolated by vacuum filtration, washed with ethanol (3×50 ml), and dried in vacuum oven at 50° C. over a weekend to obtain solifenacin succinate (“SLF-Suc,” 78.94 g) crystalline Form I.

### Example 2

### General Procedure for SLF-Succinate Evaporation

SLF-succinate Form I (100 mg) was dissolved at room temperature in a solvent. The solvent was evaporated and the residue dried in a vacuum oven at 50° C. overnight to obtain solifenacin succinate crystalline Form I. The experiments and results are summarized in Table 1.

TABLE 1

| | |
|---|---|
| Vol. (ml/g of | PXRD |

| Solvent | SLF-suc) | sample | Results |
|---|---|---|---|
| EtOH absolute | 40 | dry | Form I |
| EtOH 95% | 10 | dry | Form I |
| MeOH | 5 | dry | Form I |
| 1-Propanol | 35 | dry | Form I |
| THF | 30 | dry | Form I |
| Dioxane | 35 | dry | Form I |
| Ethyl lactate | 15 | dry | Form I + |
| | | | ~40% amorphous |
| DCM | 5 | dry | Form I |
| 1,2- | 10 | dry | Form I |
| Dichloroethane | | | |
| ACN | 30 | dry | Form I |
| DMA | 5 | dry | Form I |
| DMF | 5 | dry | Form I |

### Example 3

### General Procedure for Crystallization

SLF-succinate (500 mg) was dissolved in a solvent by heating to reflux temperature. The solution was cooled to room temperature (in some cases filtration was done before cooling), and precipitation occurred. The product was isolated by filtration and dried in a vacuum oven at 50° C. overnight to obtain solifenacin succinate crystalline Form I. The experiments and results are summarized in Table 2.

TABLE 2

| | |
|---|---|
| Vol. (ml/g of | PXRD |

| Solvent | SLF-suc) | sample | Results |
|---|---|---|---|
| t-BuOH | 15 | dry | Form I |
| IPA | 10 | dry | Form I |
| EA | 45 | dry | Form I |
| MEK | 15 | dry | Form I |
| ACN | 15 | dry | Form I |
| 2-BuOH | 10 | dry | Form I |
| toluene | 50 | wet | Form I |
| | | dry | Form I |
| Dioxane | 5 | wet | Form I |
| | | dry | Form I |
| 1-propanol | 5 | wet | Form I |
| | | dry | Form I |
| CCl₄ | 50 | wet | Form I |
| | | dry | Form I |
| EtOH | 7.5 | dry | Form I |
| THF | 10 | wet | Form I |
| | | dry | Form I |
| MTBE | 50 | wet | Form I |
| | | dry | Form I |
| acetone | 25 | dry | Form I |
| diisopropyl ether | 50 | wet | Form I |
| | | dry | Form I |
| methyl acetate | 50 | dry | Form I |

### Example 4

### Procedure for the Preparation of Form I of Solifenacin Succinate

SLF (3.22 g) was dissolved in MEK (30 ml) at room temperature. Then succinic acid (1.1 g) was added. The reaction mixture was stirred at room temperature for 18 hrs, during which it became slurry. The product was isolated by vacuum filtration, washed with methylethylketone (2×5 ml), and dried in a vacuum oven at 50° C. overnight to obtain solifenacin succinate crystalline Form I (1.33 g, 31% yield).

### Example 5

### Procedure for the Preparation of Form I of Solifenacin Succinate

SLF (2.68 g) was dissolved in EPA (30 ml) at room temperature. Then succinic acid (1 g) was added. The reaction mixture was stirred at room temperature for 19 hrs, during which it became slurry. The product was isolated by vacuum filtration, washed with EPA (2×3 ml), and dried in a vacuum oven at 50° C. overnight to obtain solifenacin succinate crystalline Form I (1.5 g, 42% yield).

### Example 6

### Procedure for the Preparation of Form I of Solifenacin Succinate

SLF (3.3 g) was dissolved in IBA (30 ml) at room temperature. Then succinic acid (1.1 g) was added. During the addition, the solution was stirred at room temperature for 3 hrs, during which it became slurry. The product was isolated by vacuum filtration, and dried in a vacuum oven at 50° C. overnight to obtain solifenacin succinate crystalline Form I (1.02 g, 23% yield).

### Example 7

### Procedure for the Preparation of Form I of Solifenacin Succinate

A vial was loaded with 2 ml solution of solifenacin succinate in MeOH (4 g in 16 ml), and then allowed to stand in another bigger vial which was loaded with another solvent at its bottom. The bigger vial was closed in order to form a solvent vapor environment. After one month, the solvent (if any) was removed with pipette, and the solid was isolated to obtain solifenacin succinate crystalline Form I. The experiments and results are summarized in Table 3.

TABLE 3

| | XRD |
|---|---|
| Solvent | Results |
| Toluene | Form I |
| IPE | Form I |
| MeOAc | Form I |
| 1-propanol | Form I |
| 2-butanol | Form I |
| ACN | Form I |
| THF | Form I |

### Example 8

### Procedure for the Preparation of Form I of Solifenacin Succinate

Solifenacin succinate (0.5 g) was dissolved in a solvent (1-2 ml) to obtain a solution. The solifenacin succinate solution was combined with an anti-solvent (20-25 ml) according to Table 7. The mixture was stirred for 3-18 hours. The product was isolated by vacuum filtration, and dried in vacuum oven at 50° C. for 18-24 hours to obtain solifenacin succinate crystalline Form I. The experiments and results are summarized in Table 4.

TABLE 4

| | Vol. | | Vol. | | |
|---|---|---|---|---|---|
| | (ml/g of | anti- | (ml/g of | | XRD results |
| solvent | SLF-suc) | solvent | SLF-suc) | Condition of anti-solvent addition | (dry) |
| MeOH | 2 | Acetone | 40 | Addition of anti-solvent to solution at | Form I |
| | | | | reflux and cooling to −5° C. | |
| MeOH | 2 | MTBE | 40 | Addition of solution to anti-solvent | Form I |
| MeOH | 2 | MeOAc | 40 | Addition of solution to cooled anti- | Form I |
| | | | | solvent at −5° C. | |
| DMSO | 4 | MTBE | 50 | Addition of anti-solvent to solution | Form I |
| DMSO | 4 | IBA | 50 | Addition of solution to cooled anti- | Form I |
| | | | | solvent at −5° C. | |
| DCM | 2 | 2- | 50 | Addition of anti-solvent to solution | Form I |
| | | BuOH | | | |
| DCM | 2 | MEK | 50 | Addition of solution to anti-solvent | Form I |
| DCM | 2 | acetone | 50 | Addition of solution to cooled anti- | Form I |
| | | | | solvent at −5° C. | |
| DCM | 2 | IPE | 50 | Addition of anti-solvent to solution | Form I |
| DCM | 2 | CCl4 | 50 | Addition of anti-solvent to solution | Form I |
| DCM | 2 | toluene | 50 | Addition of solution to anti-solvent | Form I |
| DCM | 2 | EtOAc | 50 | Addition of hot solution to anti-solvent | Form I |
| DCM | 2 | Hexane | 50 | Addition of hot solution to anti-solvent | Form I |

When it is not mentioned, the temperature is RT.

### Example 9

### Procedure for the Preparation of Form I of Solifenacin Succinate

Solid solifenacin (2 g) was dissolved in EtOH (2 ml) and MIBK (5 ml) to obtain clear solution at room temperature. Succinic acid (0.78 g) was added and the mixture was heated to 50° C. and stirred for 40 min, slurry was obtained. The mixture was cooled to room temperature and stirred over night. The product was isolated by vacuum filtration, washed with MIBK (20 ml), and dried in vacuum oven at 55° C. over night to obtain solifenacin succinate crystalline Form I (1.7 g, 64% yield).

### Example 10

### Procedure for the Preparation of Form I of Solifenacin Succinate

An oily solifenacin (4 g) was dissolved in EtOH (4 ml) and MIBK (20 ml) to obtain clear solution at 50° C. Succinic acid (1.43 g) was added and the mixture was stirred for 20 min, slurry was obtained. The mixture was cooled to room temperature and stirred over night. The product was isolated by vacuum filtration, washed with MIBK (20 ml) and dried in vacuum oven at 55° C. over night to obtain solifenacin succinate crystalline Form I (3.69 g, 70% yield).

### Example 11

### Procedure for the Preparation of Form I of Solifenacin Succinate

An oily solifenacin (3 g) was dissolved in EtOH (3 ml) and MEK (15 ml) to obtain clear solution at 50° C. Succinic acid (1.07 g) was added and the mixture was stirred for 40 min, slurry was obtained. The mixture was cooled to room temperature and stirred over night. The product was isolated by vacuum filtration, washed with MEK (20 ml) and dried in vacuum oven at 55° C. over night to obtain solifenacin succinate crystalline Form I (2.5 g, 63% yield).

### Example 12

### Procedure for the Preparation of Form I of Solifenacin Succinate

An oily solifenacin (3 g) was dissolved in EtOH (3 ml) and acetone (15 ml) to obtain clear solution at 50° C. Succinic acid (1.07 g) was added and the solution was cooled to room temperature and stirred over night. Seeding was added and the mixture was stirred for 8 hours. The product was isolated by vacuum filtration, washed with acetone (15 ml) and dried in vacuum oven at 55° C. over night to obtain solifenacin succinate crystalline Form I (2.15 g, 52% yield).

### Example 13

### Procedure for the Preparation of Form I of Solifenacin Succinate

An oily solifenacin (2 g) was dissolved in MIBK (20 ml) to obtain clear solution at 50° C. Succinic acid (0.71 g) was added and the mixture was stirred for 30 min, slurry was obtained. The mixture was cooled to room temperature and stirred 6.5 hours. The product was isolated by vacuum filtration, washed with MIBK (20 ml) and dried in vacuum oven at 55° C. over night to obtain solifenacin succinate crystalline Form I (1.6 g, 60% yield).

### Example 14

### Procedure for the Preparation of Form I of Solifenacin Succinate

An oily solifenacin (1.7 g) was dissolved in acetone (15 ml) to obtain clear solution at 50° C. Succinic acid (0.6 g) was added and slurry was obtained. The mixture was cooled to room temperature and stirred 4.5 hours. The product was isolated by vacuum filtration, washed with acetone (15 ml) and dried in vacuum oven at 55° C. over night to obtain solifenacin succinate crystalline Form I (1.32 g, 59% yield).

### Example 15

### Procedure for the Preparation of Form I of Solifenacin Succinate

An oily solifenacin (3.3 g) was dissolved in MEK (17 ml) to obtain clear solution at 50° C. Succinic acid (1.18 g) was added and slurry was obtained. The mixture was cooled to room temperature and stirred 4.5 hours. The product was isolated by vacuum filtration, washed with MEK (15 ml) and dried in vacuum oven at 55° C. over night to obtain solifenacin succinate crystalline Form I (3.1 g, 71% yield).

### Example 16

### Procedure for the Preparation of Form I of Solifenacin Succinate

Solid solifenacin (4.35 g) was dissolved in dimethoxypropane (5 ml) to obtain clear solution at 50° C. Succinic acid (1.7 g) was added and stirred for 35 min, slurry was obtained. The mixture was cooled to room temperature and stirred over night. The product was isolated by vacuum filtration, washed with dimethoxypropane (20 ml) and dried in vacuum oven at 55° C. over night to obtain solifenacin succinate crystalline Form I (4 g, 70% yield).

### Example 17

### Procedure for the Preparation of Form I of Solifenacin Succinate

Solid solifenacin (3 g) was dissolved in toluene (15 ml) to obtain clear solution at 50° C. Succinic acid (1.07 g) was added and the mixture was stirred for 20 min, slurry was obtained. The mixture was cooled to room temperature and stirred over night. The product was isolated by vacuum filtration, washed with toluene (10 ml) and dried in vacuum oven at 55° C. over night to obtain solifenacin succinate crystalline Form I (4.08 g, 102% yield).

### Example 18

### Procedure for the Preparation of Form I of Solifenacin Succinate

Solid solifenacin (3 g) was dissolved in ACN (15 ml) to obtain clear solution at 50° C. Succinic acid (1.07 g) was added and the mixture was stirred for 20 min. The mixture was cooled to room temperature and stirred over night. Seeding was done with amorphous solifenacin succinate, the mixture was cooled in ice-bath for 2 hours to obtain slurry. The product was isolated by vacuum filtration, washed with ACN (10 ml) and dried in vacuum oven at 55° C. over night to obtain solifenacin succinate crystalline Form I (1.86 g, 47% yield).

### Example 19

### Procedure for the Preparation of Form I of Solifenacin Succinate

Solid solifenacin (2 g) was dissolved in DMC (10 ml) to obtain clear solution at 50° C. Succinic acid (0.72 g) was added and the mixture was stirred for 20 min, slurry was obtained. The mixture was cooled to room temperature and stirred for 4 hrs. The product was isolated by vacuum filtration, washed with DMC (10 ml) and dried in vacuum oven at 55° C. over night to obtain solifenacin succinate crystalline Form I (1.98 g, 75% yield).

### Example 20

### Procedure for the Preparation of Form I of Solifenacin Succinate

A vial was loaded with amorphous solifenacin succinate (0.2 g) and organic solvent (1 ml) and stirred with magnetic stirrer. The mixture was stirred at 100° C. for 0.5 hrs (In the case of Acetone it was heated to 60° C.) then cooled to RT during 0.5 hrs. The mixture was kept at 4° C. for one day, then stirred at a vortex for ˜1 min and the solvent was taken out by decantation. The wet solid was kept at 4° C. The experiments and results are summarized in Table 5.

### Table 5

| | Description of | Stirring | |
|---|---|---|---|
| Solvent | procedure | Temp. (° C.) | XRD Results |
| MeOAc | clear solution | 100° C. | Form I |
| | was obtained | | |
| IsoBuAcetate | slurry | 100° C. | Form I |
| Acetone | clear solution | 60° C. | Form I |
| | was obtained | | |
| Toluene | slurry | 100° C. | Form I* |
| DEC | slurry | 100° C. | Form I |
| MTBE | slurry | 100° C. | Form I |
| CCl₄ | slurry | 100° C. | Form I* |
| Cyclohexane | slurry | 100° C. | Form I |
| petrol ether 60-80° C. | slurry | 100° C. | Form I |
| EtOAc | slurry | 100° C. | Form I |
| Methylisobutylketone | slurry | 100° C. | Form I |
| Isopropylmethyl ketone | clear solution | 100° C. | Form I |
| | was obtained | | |
| Heptane | slurry | 100° C. | Form I |
| ethyl lactate | slurry | 100° C. | Form I |

*low crystallinity, contains amorphous solifenacin succinate

### Example 21

### Procedure for the Preparation of Form I of Solifenacin Succinate

Solid solifenacin (1.6 g) was mixed with succinic acid (0.57 g) for one hour, then cyclohexane (16 ml) was added and the mixture was stirring at room temperature over night. The product was isolated by vacuum filtration, washed with cyclohexane and dried in vacuum oven at 55° C. for 24 hours to obtain solifenacin succinate crystalline Form I (0.86 g, 40% yield).

### Example 22

### Procedure for the Preparation of Form I of Solifenacin Succinate

A solid solifenacin (1.6 g) was mixed with succinic acid (0.57 g) for one hour, then heptane (16 ml) was added and the mixture was stirring at room temperature over night. The product was isolated by vacuum filtration, washed with heptane and dried in vacuum oven at 55° C. for 24 hours to obtain solifenacin succinate crystalline Form I (0.6 g, 28% yield).

### Example 23A

### Procedure for the Preparation of Form I of Solifenacin Succinate

Solid solifenacin (1.6 g) was mixed with succinic acid (0.57 g) for one hour, then n-BuOH (16 ml) was added and the mixture was stirring at room temperature over night. The product was isolated by vacuum filtration, washed with n-BuOH and dried in vacuum oven at 55° C. for 24 hours to obtain solifenacin succinate crystalline Form I (1 g, 47% yield).

### Example 23B

### Procedure for the Preparation of Form I of Solifenacin Succinate

Succinic acid (1.62 g, 1 eq) was added to SLF base (5 g). The obtained mixture was stirred at room temperature for 5 hrs to obtain SLF-succinate crystalline form I.

### Example 24

### Procedure for the Preparation of Forms I and II of Solifenacin Succinate

A vial was loaded with amorphous solifenacin succinate (0.1 g) and organic solvent (1 ml) and stirred with magnetic stirrer for 1 hr at RT. The mixture was kept at 4° C. for 5 days, then stirred at a vortex for ˜1 min and the solvent was taken out by decantation. The wet solid was kept at 4° C. The experiments and results are summarized in Table 6.

TABLE 6

| Solvent | XRD Results |
|---|---|
| DEC | Form I |
| MTBE | Form I |
| n-BuOH | Form II |
| Toluene | Form I |
| Methylethylketone | Form I |
| Acetone | Form I + II |
| Dioxane | Form I + II |
| DEE | Form I |
| Isobutylacetate | Form II |
| EtOAc | Form I |
| IPA | Form I |
| Methylisobutylketone | Form I |
| DMC | Form I |

### Example 25

### Preparation of Amorphous Form of Solifenacin Succinate by Spray Drying

5 g solifenacin succinate was dissolved in 20 ml methanol and pumped into a spray dryer. The nitrogen inlet temperature was 150° C., and the outlet temperature was 92-94° C. The amorphous form of solifenacin succinate was obtained and characterized by PXRD.

### Example 26

### Preparation of Amorphous Form of Solifenacin Succinate by Spray Drying

5 g solifenacin succinate was dissolved in 20 ml water and pumped into a spray dryer. The nitrogen inlet temperature was 100° C., and the outlet temperature was 56-70° C. The amorphous form of solifenacin succinate was obtained and characterized by PXRD.

### Example 27

### Preparation of Amorphous Form of Solifenacin Succinate by Freeze Drying

1 g solifenacin succinate was dissolved in 6 ml of water. The solution was frozen by liquid nitrogen and kept in the freezer over night, then dried by freeze dryer for 20 hours to obtain amorphous solifenacin succinate, which was kept in the refrigerator.

### Example 28

### Procedure for the Preparation of Form II of Solifenacin Succinate

SLF (3.2 g) was dissolved in methylacetate (30 ml) at room temperature. Then succinic acid (1.1 g) was added, and the solution became slurry. After 3.5 hrs, the product was isolated by vacuum filtration, washed with methylacetate (2×5 ml), and dried in a vacuum oven at 50° C. overnight to obtain solifenacin succinate crystalline Form II (2.94 g, 69% yield).

### Example 29

### Procedure for the Preparation of Form II of Solifenacin Succinate

SLF (3.26 g) was dissolved in MTBE (45 ml) at room temperature. Then succinic acid (1.1 g) was added, and the solution became slurry. After 4 hrs, the product was isolated by vacuum filtration, washed with MTBE (2×5 ml), and dried in a vacuum oven at 50° C. overnight to obtain solifenacin succinate crystalline Form II (3.31 g, 76.6% yield).

### Example 30

### Procedure for the Preparation of Form I of Solifenacin Succinate

A vial was loaded with amorphous solifenacin succinate (0.4 g), and then allowed to stand in another bigger vial loaded with a solvent at its bottom. The bigger vial was closed in order to form a solvent vapor environment. After one month, the solvent (if any) was removed with pipette, and the solid was isolated to obtain solifenacin succinate crystalline Form I. The experiments and results are summarized in Table 7.

TABLE 7

| Solvent | XRD Results |
|---|---|
| cyclohexane | Form I |
| IPA | Form I |
| EtOH | Form I |
| n-BuOH | Form I |
| Diethyl Ether | Form I |
| MTBE | Form I + Amorphous |
| THF | Form I |
| EtOAc | Form I |
| BuOAc | Form I |
| Acetone | Form I |
| MIBK | Form I |

1. A process for preparing the crystalline solifenacin succinate characterized by PXRD peaks at about 3.9, 11.2, 14.3, and 18.8°±0.2° 2θ, comprising combining solifenacin, succinic acid, and a solvent selected from the group consisting of C₃-C₅ carbonate, acetonitrile, dimethoxypropane, C₆-C₉ aromatic hydrocarbon, diethyl ether, diisopropyl ether, C₅-C₉ ester, C₁-C₄ alcohol, C₃-C₉ ketone, cyclohexane, heptane, and mixtures thereof to obtain the crystalline form.

2. (canceled)

3. The process of claim 1, wherein the C₆-C₉ aromatic hydrocarbon is toluene, the C₃-C₅ carbonate is dimethyl carbonate, the C₁-C₄ alcohol is selected from the group consisting of ethanol, isopropyl alcohol and n-butanol, the C₃-C₉ ketone is selected from the group consisting of methylethylketone, acetone, and methyl isobutylketone, the C₅-C₉ ester is isobutyl acetate.

4. The process of claim 1, wherein the solvent is selected from the group consisting of ethanol, isobutyl acetate, methylethylketone, isopropylether, methyl isobutylketone, acetone, a mixture of ethanol and methylethylketone, a mixture of ethanol and methyl isobutylketone, and a mixture of ethanol and acetone.

5. The process of claim 1, comprising the steps of:
a) combining solifenacin with the solvent to form a solution; and
b) adding succinic acid to the solution to obtain the crystalline form.

6. The process of claim 5, wherein a slurry containing solifenacin succinate is obtained after the succinic acid is added.

7. The process of claim 5, further comprising heating the mixture of solifenacin and solvent after they are combined in step (a).

8. The process of claim 7, wherein the heating is to a temperature of about 30° C. to about 70° C.

9. The process of claim 8, wherein the heating is to a temperature of about 40° C. to about 60° C.

10. The process of claim 9, wherein the heating is to a temperature of about 50° C.

11. The process of claim 7, wherein a clear solution is obtained after heating.

12. The process of claim 5, further comprising cooling the reaction mixture after succinic acid is added in step (b).

13. The process of claim 12, wherein the cooling is to a temperature of about room temperature to about −10° C.

14. The process of claim 13, wherein the cooling is to a temperature of about 0° C.

15. The process of claim 14, wherein the reaction mixture is stirred.

16. The process of claim 3, wherein the solvent is selected from the group consisting of isopropanol, isobutyl acetate, and methylethylketone.

17. The process of claim 16, wherein solifenacin and the solvent are combined at a temperature of about 15° C. to about 30° C.

18. The process of claim 16, wherein the reaction mixture is stirred to obtain a slurry.

19. The process of claim 18, wherein the stirring is stirring is at a temperature of about 15° C. to about 30° C.

20. The process of claim 14, comprising the steps of:
a) combining solifenacin with the succinic acid; and
b) precipitating the crystalline form.

21. The process of claim 20, further comprising combining the result of step (a) with a solvent selected from the group consisting of cyclohexane, heptane, and n-butanol.

22. The process of claim 20, wherein the reaction mixture is stirred.

23. The process of claim 22, further comprising the step of recovering the precipitated crystalline solifenacin succinate.

24. A process for preparing the crystalline solifenacin succinate characterized by PXRD peaks at about 3.9, 11.2, 14.3, and 18.8°±0.2° 2θ, comprising slurrying amorphous solifenacin succinate in a solvent selected from the group consisting of heptane, petroleum ether, cyclohexane, methyl t-butyl ether, ethanol, methyl isobutylketone, CCl₄, toluene, diethyl carbonate, ethyl lactate, isobutyl acetate, methylethylketone, diethyl ether, isopropanol, dimethyl carbonate, and mixtures thereof.

25. The process of claim 24, wherein the solvent is isobutyl acetate and the slurry is heated to a temperature of more than room temperature.

26. The process of claim 24, wherein the ratio of solvent to solifenacin succinate is from about 4:1 to about 10:1 ml/g.

27. The process of claim 25, wherein the ratio of solvent to solifenacin succinate is about 5:1.

28. The process of claim 24, wherein the mixture of the solifenacin succinate and the solvent is stirred at about room temperature to about 110° C.

29. The process of claim 28, wherein the stirring is at about 100° C.

30. The process of claim 24, wherein, after the heating step, the mixture of the solifenacin succinate and the solvent is stirred at about 15° C. to about 30° C.

31. The process of claim 23, wherein the solvent is removed.

32. The process of claim 31, wherein the solvent is removed by decantation.

33. The process of claim 23, further comprising the step of recovering the crystalline solifenacin succinate.

34. A process for preparing the crystalline solifenacin succinate characterized by PXRD peaks at about 3.9, 11.2, 14.3, and 18.8°±0.2° 2θ, comprising combining solifenacin succinate with a solvent selected from the group consisting of ethanol, methanol, 1-propanol, tetrahydrofuran, dioxane, ethyl lactate, dichloromethane, 1,2-dichloroethane, acetonitrile, dimethylacetamide, dimethylformamide, t-butanol, 2-butanol, isopropanol, methylethylketone, toluene, CCl₄, methyl t-butyl ether, di-isopropyl ether, methyl acetate, ethyl acetate, acetone, isopropylmethyl ketone, and mixtures thereof to form a solution.

35. The process of claim 34, wherein the solvent is selected from the group consisting of ethanol, methanol, 1-propanol, tetrahydrofuran, dioxane, ethyl lactate, dichloromethane, 1,2-dichloroethane, acetonitrile, dimethylacetamide, dimethylformamide, and mixtures thereof, and wherein a sufficient amount of solvent is removed to obtain the crystalline solifenacin succinate.

36. The process of claim 35, wherein the ratio of solvent to solifenacin succinate is about 5:1 to about 40:1 ml/g.

37. The process of claim 36, wherein the ratio of solvent to solifenacin succinate is about 12:1 to about 40:1 ml/g.

38. The process of claim 35, wherein the solvent is removed by evaporation.

39. The process of claim 38, wherein the evaporation is performed by using an evaporator.

40. The process of claim 38, wherein the evaporation is performed at a temperature of about 20° C. to about 80° C.

41. The process of claim 40, wherein the evaporation is performed at a temperature of about 50° C.

42. The process of claim 38, wherein the evaporation is performed under vacuum.

43. The process of claim 35, wherein the solvent is ethyl lactate, and wherein the solifenacin succinate is obtained contains amorphous solifenacin succinate.

44. The process of claim 34, wherein the solvent is selected from a group consisting of methyl acetate, acetone, isopropylmethyl ketone, and mixtures thereof.

45. The process of claim 44, wherein the solution is stirred at about 60° C. to about 110° C.

46. The process of claim 45, wherein the stirring is at about 100° C.

47. The process of claim 44, wherein the solvent is removed.

48. The process of claim 47, wherein the solvent is removed by decantation.

49. The process of claim 34, comprising crystallizing solifenacin succinate from a solvent selected from the group consisting of t-butanol, isopropanol, ethyl acetate, methylethylketone, acetonitrile, 2-butanol, toluene, dioxane, 1-propanol, carbon tetrachloride, ethanol, tetrahydrofuran, methyl t-butyl ether, acetone, di-isopropyl ether, methyl acetate, methanol, dimethylsulfoxide, dichloromethane, isopropylmethylketone, and mixtures thereof.

50. The process of claim 49, wherein the solvent is selected from the group consisting of t-butanol, isopropanol, ethyl acetate, methylethylketone, acetonitrile, 2-butanol, toluene, dioxane, 1-propanol, CCl₄, ethanol, tetrahydrofuran, methyl t-butyl ether, acetone, di-isopropyl ether, methyl acetate, isopropylmethylketone, and mixtures thereof, and the process comprises dissolving solifenacin succinate in the solvent, cooling the solution, and recovering the crystalline solifenacin succinate.

51. The process of claim 50, wherein the dissolving step comprises heating the solvent.

52. The process of claim 51, wherein the heating step is to a temperature of about 50° C. to about 100° C.

53. The process of claim 50, wherein the cooling step is to a temperature of about 30° C. to about 5° C.

54. The process of claim 50, wherein the cooling step is to room temperature.

55. The process of claim 49, wherein the solvent is selected from the group consisting of methanol, dimethylsulfoxide, and dichloromethane, and the process comprises dissolving solifenacin succinate in the solvent, combining the solution with an anti-solvent selected from the group consisting of acetone, methyl t-butyl ether, methyl acetate, isobutyl acetate, 2-butanol, methylethylketone, isopropylether, carbon tetrachloride, toluene, ethyl acetate, and hexane.

56. The process of claim 55, wherein the ratio between the solvent and the solifenacin succinate is from about 1.5 to about 5 ml/g.

57. The process of claim 55, wherein the ratio between the anti-solvent and the solvent is from about 12.5 to about 25 by volume.

58. The process of claim 55, wherein the mixture of the solution and the anti-solvent is stirred.

59. The process of claim 55, wherein the solvent is methanol, and wherein the anti-solvent is selected from the group consisting of acetone, methyl t-butyl ether, and methyl acetate.

60. The process of claim 55, wherein the solvent is dimethylsulfoxide, and wherein the anti-solvent is selected from the group consisting of methyl t-butyl ether and isobutyl acetate.

61. The process of claim 55, wherein the solvent is dichloromethane, and wherein the anti-solvent is selected from the group consisting of 2-butanol, methylethylketone, acetone, isopropylether, carbon tetrachloride, toluene, ethyl acetate, and hexane.

62. The process of claim 59, wherein the anti-solvent is acetone, and wherein the solution of the dissolving step is heated prior to the combining step.

63. The process of claim 62, wherein the heating is to a temperature of about 50° C. to about reflux temperature.

64. The process of claim 63, wherein the heating is to a temperature of about reflux temperature.

65. The process of claim 62, further comprising a cooling step prior to the recovering step.

66. The process of claim 65, wherein the cooling is to a temperature of about 5° C. to about −10° C.

67. The process of claim 61, wherein the anti-solvent is selected from the group consisting of methylethylketone, toluene, ethyl acetate, and hexane, and wherein the solution is added to the anti-solvent.

68. The process of claim 67, wherein the anti-solvent is selected from the group consisting of ethyl acetate and hexane, and wherein the added solution is at a temperature higher than room temperature.

69. The process of claim 55, wherein the solvent/anti-solvent is selected from the group consisting of methanol/methyl acetate, dimethylsulfoxide/isobutyl acetate, and dichloromethane/ethyl acetate, and wherein the solution is added to a cooled anti-solvent.

70. The process of claim 69, wherein the cooled anti-solvent is at a temperature of about 5° C. to about −10° C.

71. The process of claim 34, further comprising the step of recovering the crystalline solifenacin succinate.

72. A process for preparing the crystalline solifenacin succinate characterized by PXRD peaks at about 3.9, 11.2, 14.3, and 18.8°±0.2° 2θ, comprising the step of exposing solifenacin succinate to solvent vapors for a sufficient time to form the crystalline form, wherein the solvent is selected from the group consisting of cyclohexane, isopropanol, ethano, n-butanol, diethyl ether, methyl t-butyl ether, ethyl acetate, butyl acetate, acetone, methyl isobutylketone, toluene, isopropylether, methyl acetate, 1-propanol, 2-butanol, acetonitrile, tetrahydrofuran, and mixtures thereof.

73. The process of claim 72, wherein the solifenacin succinate is dissolved in methanol.

74. The process of claim 73, wherein the solvent is selected from the group consisting of toluene, isopropylether, methyl acetate, 1-propanol, 2-butanol, acetonitrile, tetrahydrofuran, and mixtures thereof.

75. The process of claim 72, wherein the solifenacin succinate is exposed to solvent vapors in a closed container.

76. The process of claim 75, wherein the solifenacin succinate with the solvent vapors in the container is maintained for about 10 to about 40 days.

77. The process of claim 72, wherein a mixture of solvent and a solifenacin succinate precipitate is obtained in the container.

78. The process of claim 77, wherein the solvent obtained is removed.

79. The process of claim 72, further comprising isolating the crystalline solifenacin succinate.

80. A process for preparing a crystalline form of solifenacin succinate characterized by PXRD peaks at about 3.9, 11.2, 14.3, and 18.8°±0.2° 2θ, comprising combining succinic acid and solifenacin.

81. The process of claim 80, wherein the reaction mixture is stirred

82. Crystalline form of solifenacin succinate characterized by PXRD peaks at about 4.3, 14.7, and 16.2°±0.2° 2θ.

83. The crystalline form of claim 82, further characterized by PXRD peaks at about 11.7, 18.3, 19.9, 22.3, 23.7 and 25.6°±0.2° 2θ.

84. The crystalline form of claim 82 containing not more than about 10 wt % of any other crystalline form of solifenacin succinate.

85. The crystalline form of claim 82 containing not more than about 10 wt % of the crystalline form of solifenacin succinate characterized by PXRD peaks at about 3.9, 11.2, 14.3, and 18.8°±0.2° 2θ.

86. The crystalline form of claim 82 containing a total weight of all other crystalline forms of solifenacin succinate of not more than about 10 wt %.

87. Crystalline form of solifenacin succinate substantially as depicted in FIG. 4.

88. A process for preparing the crystalline solifenacin succinate of claim 82, comprising the steps of:
a) combining solifenacin with a solvent selected from methyl acetate and methyl t-butyl ether to obtain a solution; and
b) adding succinic acid to obtain a slurry containing solifenacin succinate.

89. A process according to claim 88, wherein the solifenacin and the solvent are combined at a temperature of about 15° C. to about 30° C.

90. A process according to claim 88, wherein the reaction mixture of step (b) is stirred.

91. A process according to claim 90, wherein the stirring is for about 2 to about 6 hours.

92. A process according to claim 88, further comprising the step of:
c) recovering the crystalline solifenacin succinate.

93. A process for preparing the crystalline solifenacin succinate of claim 82, comprising the step of slurrying amorphous solifenacin succinate in a solvent selected from the group consisting of n-butanol and isobutyl acetate.

94. The process of claim 93, wherein the slurry is stirred for about 0.5 to about 3 hours.

95. The process of claim 94, wherein the stirring is at about room temperature.

96. A process according to claim 93, further comprising the step of recovering the crystalline solifenacin succinate.

97. A process for preparing a mixture of the crystalline solifenacin succinate characterized by PXRD peaks at about 3.9, 11.2, 14.3, and 18.8°±0.2° 2θ and the crystalline solifenacin succinate of claim 82, comprising the step of slurrying amorphous solifenacin succinate in a solvent selected from acetone and dioxane.

98. The process of claim 97, wherein the slurry is stirred for about 0.5 to about 3 hours.

99. A process according to claim 97, further comprising the step of recovering the crystalline solifenacin succinate.

100. Amorphous form of solifenacin succinate.

101. Amorphous form of solifenacin succinate characterized by a PXRD pattern substantially as depicted in FIG. 2 or FIG. 3.

102. Amorphous form of claim 100 containing not more than about 10 wt % of any crystalline form of solifenacin succinate.

103. The amorphous form of claim 100 containing not more than about 10 wt % of the crystalline form of solifenacin succinate characterized by PXRD peaks at about 3.9, 11.2, 14.3, and 18.8°±0.2° 2θ.

104. The amorphous form of claim 100 containing a total weight of all crystalline solifenacin succinate of not more than about 10 wt %.

105. A process for preparing the amorphous solifenacin succinate of a claim 100, comprising the steps of:
a) dissolving solifenacin succinate in methanol or water to form a solution; and
b) spray-drying the solution to obtain amorphous solifenacin succinate.

106. The process of claim 105, wherein the solution is spray-dried at an inlet temperature of from about 30° C. to about 200° C.

107. The process of claim 105, wherein the solution is spray-dried at an inlet temperature of from about 50° C. to about 150° C.

108. The process of claim 105, wherein the solution is spray-dried at an outlet temperature of from about 50° C. to about 100° C.

109. The process of claim 105, wherein the solvent is methanol, the inlet temperature is about 150° C., and the outlet temperature is from about 92° C. to about 94° C.

110. The process of claim 105, wherein the solvent is water, the inlet temperature is about 100° C., and the outlet temperature is from about 56° C. to about 70° C.

111. The process of claim 105, wherein the drying gas is selected from a group consisting of nitrogen, nitrogen-enriched air, and argon.

112. A process according to claim 105, further comprising the step of recovering the amorphous solifenacin succinate.

113. A process for preparing the amorphous solifenacin succinate of claim 100, comprising the steps of:
a) dissolving solifenacin succinate in water; and
b) lyophilizing solifenacin succinate from water.

114. The process of claim 113, wherein the lyophilization is at a temperature of about −20° C. to about −50° C.

115. The process of claim 113, wherein the lyophilization is for about 15 to about 30 hours.

116. A pharmaceutical composition comprising at least one of the amorphous solifenacin succinate of claim 100 and at least one pharmaceutically acceptable excipient.

117. A process for preparing the pharmaceutical composition of claim 116, comprising combining the amorphous solifenacin succinate with the pharmaceutically acceptable excipient.

118. (canceled)

119. A method of treatment of overactive bladder syndrome, comprising administering crystalline solifenacin succinate of claims 82.

120. A pharmaceutical composition comprising the crystalline solifenacin succinate of claim 82 and at least one pharmaceutically acceptable excipient.

121. A process for preparing the pharmaceutical composition of claim 120, comprising combining the crystalline solifenacin succinate with the pharmaceutically acceptable excipient.

122. A method of treatment of overactive bladder syndrome, comprising administering amorphous solifenacin succinate of claim 100.

123. The process of claim 3, wherein the solvent is a mixture of toluene and acetone.
